(19) United States
(12) Patent Application Publication
(10) Pub. No.: US 2020/0063211 A1
(43) Pub. Date: Feb. 27, 2020

(54) METHOD OF DISTINGUISHING LIPOSARCOMA FROM NON-LIPOSARCOMA

(71) Applicant: H. LEE MOFFITT CANCER CENTER AND RESEARCH INSTITUTE, INC., TAMPA, FL (US)

(72) Inventors: ANTHONY M. MAGLIOCCO, TAMPA, FL (US); SONER ALTIOK, TAMPA, FL (US); EVITA HENDERSON-JACKSON, BRANDON, FL (US); YIN XIONG, TAMPA, FL (US)

(21) Appl. No.: 16/487,662

(22) PCT Filed: Feb. 28, 2018
(86) PCT No.: PCT/US2018/020279

§ 371 Date: Aug. 21, 2019

Related U.S. Application Data
(60) Provisional application No. 62/464,875, filed on Feb. 28, 2017.

(51) Int. Cl. C12Q 1/6886; G16H 50/30; C40B 30/04; C12Q 1/6827

(52) U.S. Cl. CPC C12Q 1/6886; G16H 50/30; C12Q 2600/158; C12Q 1/6827; C40B 30/04

(57) ABSTRACT

The present invention concerns materials and methods useful for distinguishing between liposarcoma and non-liposarcoma. The invention further includes methods for treating a patient having a lesion from which a sample has been analyzed. The invention also includes arrays useful for distinguishing between liposarcoma and non-liposarcoma.

### CROSS-REFERENCE TO RELATED APPLICATION

The present application claims the benefit of U.S. Provisional Application Ser. No. 62/464,875, filed Feb. 28, 2017, which is hereby incorporated by reference herein in its entirety, including any figures, tables, nucleic acid sequences, amino acid sequences, or drawings.

### FIELD OF INVENTION

This invention relates to tumorigenic assays. Specifically, the invention provides a method of distinguishing between liposarcoma and non-liposarcoma using an 18 gene signature.

### BACKGROUND OF THE INVENTION

Liposarcoma is the most common adult sarcoma, accounting for 15% to 25% of all sarcomas [1]. Liposarcoma encompass five types: atypical lipomatous tumor/well-differentiated liposarcoma (ALT/WDL), dedifferentiated liposarcoma (DL), myxoid liposarcoma (ML), pleomorphic liposarcoma (PL) and liposarcoma, not otherwise specified (NOS). The World Health Organization (WHO) categorizes atypical lipomatous tumor/well-differentiated liposarcoma as a locally aggressive neoplasm with no potential for metastasis unless it undergoes dedifferentiation. While dedifferentiated liposarcoma, myxoid liposarcoma, pleomorphic liposarcoma and liposarcoma, not otherwise specified are categorized as malignant due to metastatic potential [2].

ALT/WDL resides in the abdominal cavity or in an arm or leg and presents as a large painless mass. This type of liposarcoma is a less aggressive subtype. DL is a more aggressive version of the WDL however this type of liposarcoma is less aggressive than sarcomas identified as “high-grade”. ML is a common form of liposarcoma which occurs in the leg with a high risk of recurrence in other soft tissue sites or in bones, such as those in the spine and pelvis. PL is the least common form of liposarcoma that affects and arm or leg. This type of liposarcoma is often more aggressive than other liposarcomas and often spreads to other sites in the body such as the lung and into soft tissue.

Diagnostic discordance still occurs even among expert soft tissue pathologists despite advanced molecular testing. Increasing use of smaller biopsy specimens for diagnosis including overlapping histologic features, compounds the difficulty in diagnosis.

### BRIEF SUMMARY OF THE INVENTION

Due to overlapping histological features and tumors arising on a variety of anatomic locations, there is still a significant diagnostic discordance in identifying liposarcomas, even among expert soft tissue pathologists. With the increasing use of smaller biopsy specimens for diagnosis, tissue samples are at times insufficient for current molecular testing.

The inventors sought to develop a gene expression signature unique to liposarcoma (subtype non-specific) to serve as a diagnostic tool to distinguish liposarcoma from other cancers (non-liposarcomas).

The inventors have developed a novel gene expression signature for liposarcoma (subtype non-specific) to serve as an adjunct diagnostic tool to distinguish between liposarcoma versus non-liposarcoma among lipomatous lesions in order to improve upon diagnosing liposarcoma.

Accordingly, one aspect of the invention is a diagnostic method for distinguishing between liposarcoma and non-liposarcoma in a patient. Another aspect of the invention method for treating a malignancy in a patient, comprising carrying out the diagnostic method and administering a treatment to the patient appropriate for liposarcoma if the composite gene signature score is indicative of liposarcoma, or administering a treatment to the patient appropriate for a non-liposarcoma if the composite gene signature is indicative of non-liposarcoma. Another aspect of the invention concerns arrays useful for carrying out the diagnostic methods and treatment methods of the invention.

The arrays and diagnostic method of the invention may be used as an adjunct diagnostic tool for distinguishing liposarcomas from other sarcomas based on a expression signature. This novel technology is a test developed by pathologists to be added to their current diagnostic algorithms when they are asked by clinicians to determine the type of sarcoma that has been biopsied. It can be used in addition to immunohistochemistry (IHC) tests to give pathologists greater confidence in their results. Accurate diagnosis of liposarcoma could help clinicians decide on appropriate treatment.

### BRIEF DESCRIPTION OF THE DRAWINGS

FIG. 1 is a table depicting the 25-gene signature.

FIG. 2 is a scatterplot depicting PC1 of the 25 genes for the 50 sarcoma samples (TCC). TCC non-liposarcoma (n=31) represents the 31 non-liposarcoma samples from TCC used as part of the training dataset; TCC liposarcoma (n=19) represents the 19 liposarcoma samples from TCC used as part of the training dataset. The cutoff is PC1<0 for liposarcoma.

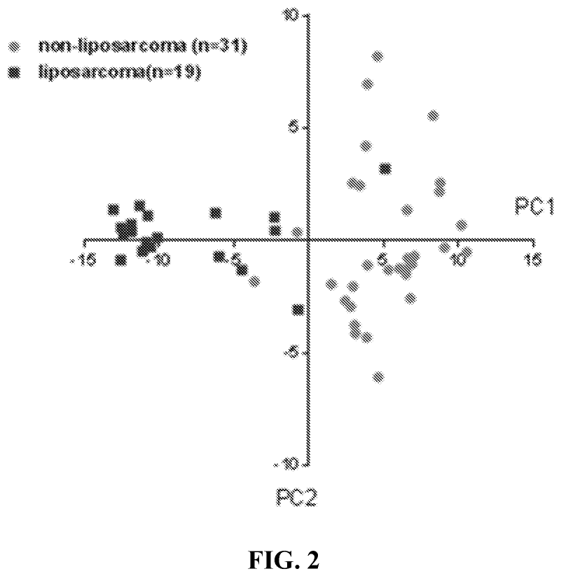

FIG. 3 is a table depicting the cutoff for liposarcoma as well as the classification accuracy for the PC1 for the 25-gene signature.

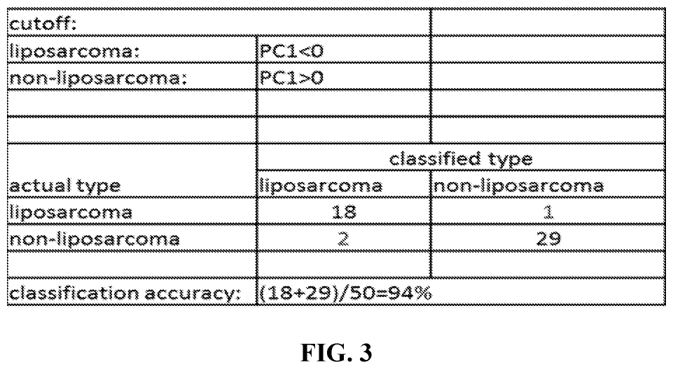

FIG. 4 is a table depicting the 18 gene signature for distinguishing liposarcoma from non-liposarcoma. Note: up/down=mean value of the gene for liposarcoma patients is higher/lower than non-liposarcoma.

FIG. 5 is a scatterplot depicting PC1 of the 18 genes for the 100 sarcoma samples (TCC and external). TCC non-liposarcoma (n=31) represents the 31 non-liposarcoma samples from TCC used as part of the training dataset; TCC liposarcoma (n=19) represents the 19 liposarcoma samples from TCC used as part of the training dataset; GSE32569 (n=12) represents paired biopsies from 6 metastatic Alveolar soft part sarcoma (ASPS) before and after Cediranib treatment; GSE12972 (n=38) represents 19 pairs of untreated primary cell cultures obtained from liposarcoma and doxorubicin treated cultures from the same liposarcoma. The cutoff is PC1<0 for liposarcoma.

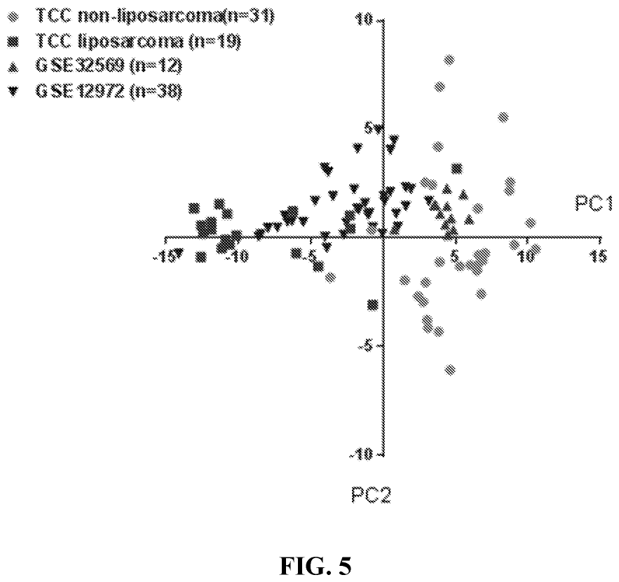

FIG. 6. ROC for the 18-gene signature on training datasets (50 samples). The ROC (Receiver operating characteristic) curve is created by plotting the true positive rate (TPR) against the false positive rate (FPR) at various threshold settings. The true-positive rate is also known as sensitivity. The false-positive rate can be calculated as (1−specificity).

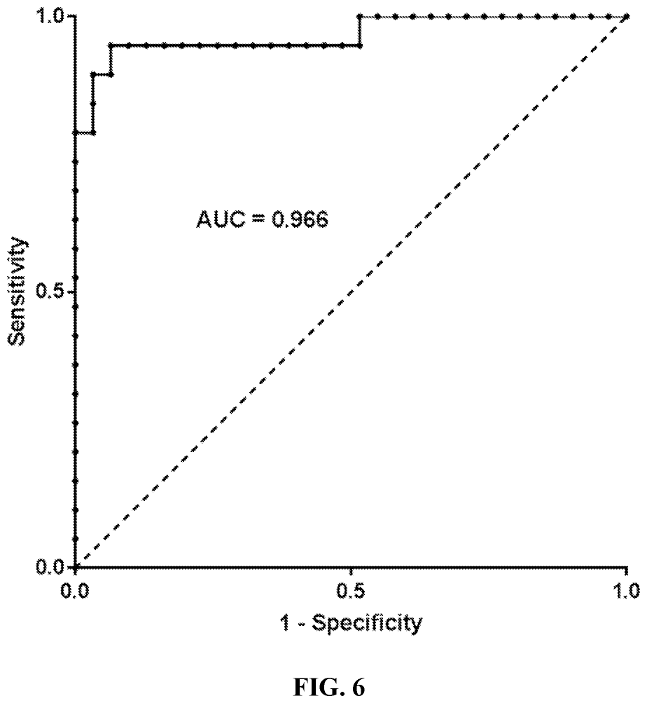

FIG. 7. ROC for the 18-gene signature on validation datasets (50 samples). The AUC (Area under curve) measures the performance of the signature. An area of 1 represents a perfect signature; an area of 0.5 represents a worthless signature.

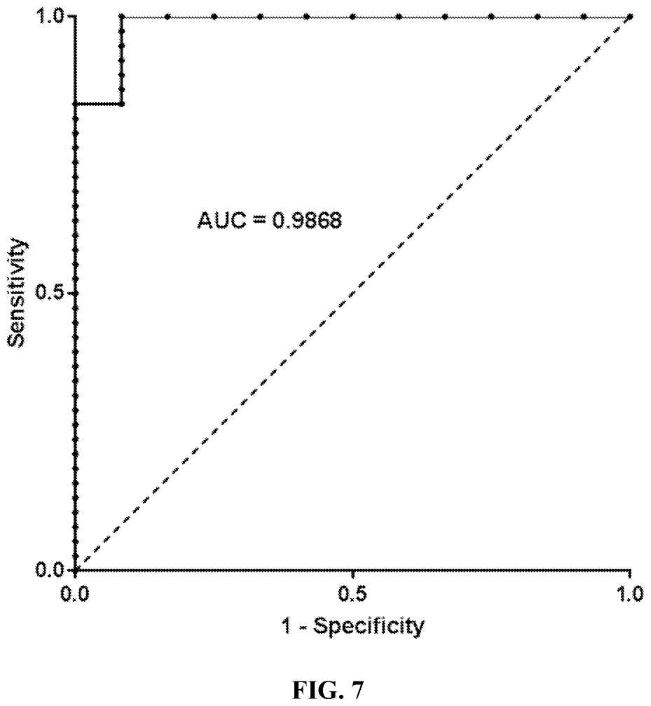

### DETAILED DESCRIPTION OF THE INVENTION

In the following detailed description of the preferred embodiments, reference is made to the accompanying drawings, which form a part hereof, and within which are shown by way of illustration specific embodiments by which the invention may be practiced. It is to be understood that other embodiments by which the invention may be practiced. It is to be understood that other embodiments may be utilized and structural changes may be made without departing from the scope of the invention.

Among the liposarcoma subtypes, each demonstrate distinct histology and biology that has been validated by molecular analysis. ALT/WDL and DL, the most frequently occurring subtypes (40-50% of all liposarcomas), contain supernumerary rings and/or giant marker chromosomes with amplified sequences originating from the 12q13-15 region resulting in amplification of MDM2 and CDK4 (in about 90% of cases) [3,4]. MLs, 15-20% of all liposarcomas, are characterized by the recurrent translocation t(12:16)(q13;p11) resulting in FUS-DDIT3 gene fusion, present in over 95% of cases [5]. PLs are very rare and possess a complex genomic profile. Of note, PLs do not possess MDM2/CDK4 amplification [6,7]. LNOS is a group that contains liposarcomas that exhibit an unusual histology revealing a combination of patterns classically a WDL histology mixed with a PL histology, but with current molecular tests some of these tumors contain MDM2 amplification and are thought to be a variant of DL with homologous lipoblastic differentiation rather than a composite tumor [1,8].

Despite current molecular techniques (e.g., Fluorescence in-situ hybridization (FISH), reverse transcription polymerase chain reaction (RT-PCR)) used to aid in the diagnosis and classification of liposarcomas, diagnostic discordance occurs even among expert soft tissue pathologists. Lipomatous tumors exhibit overlapping histologic features and may even mimic non-neoplastic lesions. It is becoming more difficult and challenging to diagnosis liposarcomas with current use of smaller and smaller biopsy specimens for initial diagnosis. At times, tissue samples are insufficient for current molecular testing. The inventors sought to develop a novel gene expression signature for liposarcoma (subtype non-specific) to serve as an adjunct diagnostic tool to distinguish between liposarcoma versus non-liposarcoma. When clinical history and radiological findings suggest a possible sarcoma in a patient with biopsy (or repeat biopsy) showing atypical adipocytes, or spindled/pleomorphic cells, or an admixture, it is clinically important to determine if the tumor is a liposarcoma versus other sarcoma infiltrating or adjacent to fat because the patient may undergo surgery or may receive chemotherapy/radiation.

Treatment for liposarcoma is challenging since complete surgical removal of tumors within the abdomen is difficult, in part because of the difficulty in getting clear margins of normal tissue. The combination of surgery and radiation therapy has been shown to prevent recurrence at the surgical site in about 85-90% of liposarcoma cases.

Past gene expression profiling studies of sarcomas have shown liposarcomas clustering with malignant fibrous histiocytoma (also known as undifferentiated pleomorphic sarcoma) and leiomyosarcoma, but these studies contained too few samples [9,10]. A liposarcoma-specific microarray study of 28 liposarcomas (11 well-differentiated, 3 dedifferentiated, 7 myxoid, 7 round cell) and eight lipomas identified through hierarchical clustering analysis clustering of dedifferentiated tumors with myxoid/round cell liposarcomas and clustering of well-differentiated liposarcoma with lipoma [11]. The investigators were not able to differentiate well-differentiated liposarcoma from lipoma. Again, the findings were likely due to limited sample numbers.

None of these prior studies have progressed from research into a clinical assay. But given advances in the gene annotation and microarray design, as well as, standardization of protocols and platforms, the use of gene expression profiling as a clinical tool is feasible [12-16]. Here, the inventors used microarray-based gene expression profiling to develop a gene signature to differentiate liposarcoma from non-liposarcoma to be used to aid in the differential diagnosis of liposarcoma and other sarcomas adjacent to or infiltrating adipose tissue.

A retrospective study was performed by the inventors on a total of 50 soft tissue sarcomas with gene expression data (HuRSTA chips) containing 19 liposarcomas and 31 non-liposarcomas. Analysis identified a set of 18 significantly differentially expressed genes (p<0.01) between liposarcomas and non-liposarcomas. Principal component analysis was used to define the cutoff (PC1<0=liposarcoma; PC1>0=non-liposarcoma). This novel 18-gene signature was self-validated on the training data set with 18 out of 19 liposarcomas classified as “liposarcoma” and 29 out of 31 non-liposarcomas classified as “non-liposarcoma” with a sensitivity of 94.74% and specificity of 93.55% (AUC=0.966).

The signature was further validated on external data sets publicly available at GEO: (a) 12 of 12 paired biopsies from 6 metastatic alveolar soft part sarcoma (ASPS) were classified as “non-liposarcoma” with a specificity of 100% and (b) 27 of 38 paired biopsies of 19 untreated primary cell cultures obtained from liposarcoma and doxorubicin treated cultures from the same liposarcoma were classified as “liposarcoma” with a sensitivity of 71.05% (AUC=0.9868). A total of 100 samples were evaluated and 86 cases were correctly classified with an overall accuracy of 86%.

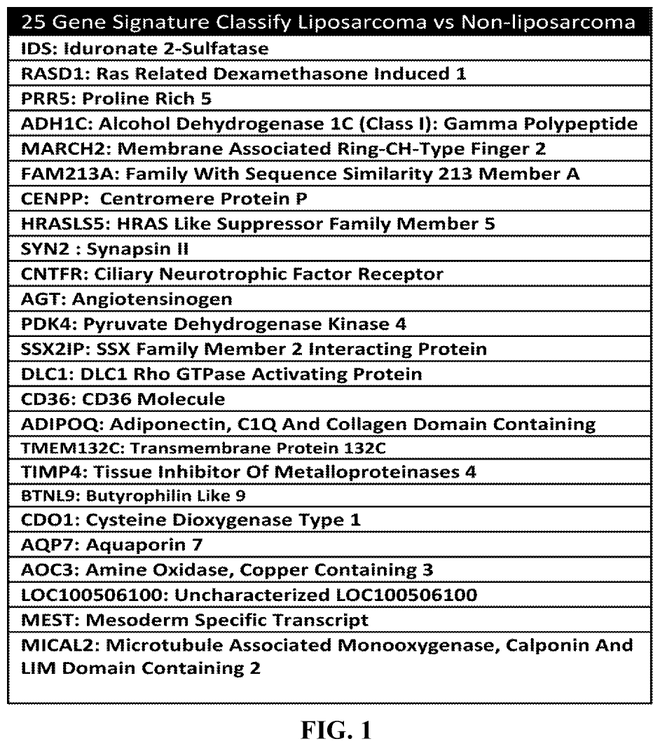

One aspect of the invention concerns a diagnostic method for distinguishing between liposarcoma and non-liposarcoma in a patient, comprising:
determining expression levels of two or more genes in a sample obtained from the subject, wherein the two or more genes comprise two or more genes from among: Iduronate 2-Sulfatase (IDS), Ras Related Dexamethasone Induced 1 (RASD1), Proline Rich 5 (PRR5), Alcohol Dehydrogenase 1C (Class I): Gamma Polypeptide (ADH1C), Membrane Associated Ring-CH-Type Finger 2 (MARCH2), Family With Sequence Similarity 213 Member A (FAM213A), Centromere Protein P (CENPP), HRAS Like Suppressor Family Member 5 (HRASLS5), Synapsin II (SYN2), Ciliary Neurotrophic Factor Receptor (CNTFR), Angiotensinogen (AGT), Pyruvate Dehydrogenase Kinase 4 (PDK4), SSX Family Member 2 Interacting Protein (SSX2IP), DLC1 Rho GTPase Activating Protein (DLC1), CD36 Molecule (CD36), Adiponectin, C1Q and Collagen Domain Containing (ADIPOQ), Transmembrane Protein 132C (TMEM132C), Tissue Inhibitor of Metalloproteinases 4 (TIMP4), Butyrophilin Like 9 (BTNL9), Cysteine Dioxygenase Type 1 (CDO1), Aquaporin 7 (AQP7), Amine Oxidase, Copper Containing 3 (AOC3), Uncharacterized LOC100506100 (LOC100506100), Mesoderm Specific Transcript (MEST), and Microtubule Associated Monooxygenase, Calponin and LIM Domain Containing 2 (MICAL2);
analyzing the determined expression levels to generate a composite gene signature score that is indicative of liposarcoma or non-liposarcoma. In some embodiments, the two or more genes comprise or consist of 2, 3, 4, 5, 6, 7, 8, 9, 10, 11, 12, 13, 14, 15, 16, 17, 18, 19, 20, 21, 22, 23, 24, or all 25 of the foregoing genes (also listed in FIG. 1).

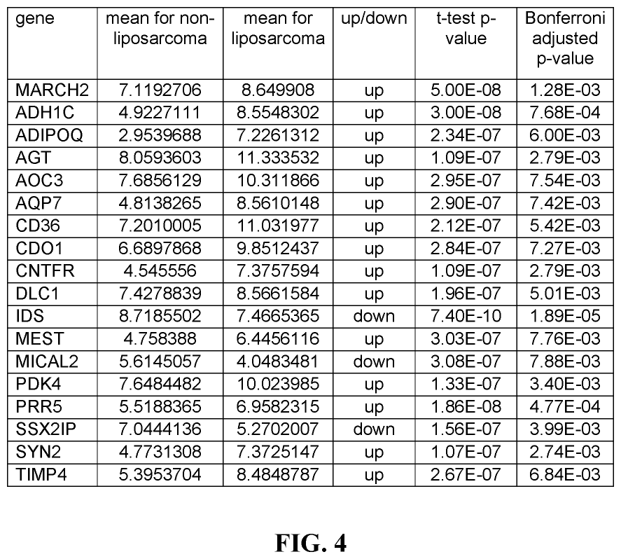

In some embodiments, the expression levels of the two or more genes in the sample comprise two or more genes selected from among MARCH2, ADH1C, ADIPOQ, AGT, AOC3, AQP7, CD36, CDO1, CNTFR, DLC1, IDS, MEST, MICAL2, PDK4, PRR5, SSX2IP, SYN2, and TIMP4. In some embodiments, the two or more genes comprise or consist of 2, 3, 4, 5, 6, 7, 8, 9, 10, 11, 12, 13, 14, 15, 16, 17, or all 18 of the foregoing genes (also listed in FIG. 4).

In some embodiments, the determining step is carried out using an array, such as an array described herein, by contacting the array with the sample.

In some embodiments, the sample is a tissue sample.

In some embodiments, the sample is from a lipomatous lesion. In some embodiments, the lipomatous lesion is a tumor.

The analyzing step may be carried out using any appropriate statistical method. In some embodiments, the statistical method is selected from among Principal Component Analysis (PCA), clustering analysis, or multivariate regression analysis to generate the composite gene signature score.

In some embodiments, the analysis comprises performing Principal Component Analysis (PCA) on the expression levels to obtain a first Principal Component score (PC1), wherein a PC1 less than zero is indicative of liposarcoma.

Data sets can be analyzed by utilizing one or more statistical methods (e.g., principle component analysis, least squares regression, linear discriminate analysis, K-nearest neighbors, logistic regression, etc.). Principal component analysis (PCA) has been used to analyze gene expression data. More generally, PCA can be used to analyze feature value data of biomarkers in order to construct a decision rule that discriminates liposarcomas and non-liposarcomas. Principal component analysis is a classical technique to reduce the dimensionality of a data set by transforming the data to a new set of variable (principal components) that summarize the features of the data. See, for example, Jolliffe, 1986, Principal Component Analysis, Springer, New York, which is hereby incorporated by reference. Principal component analysis is also described in Draghici, 2003, Data Analysis Tools for DNA Microarrays, Chapman & Hall/CRC, which is hereby incorporated by reference.

In some embodiments, the composite gene signature score is indicative of liposarcoma, and the method further comprises administering a treatment to the patient appropriate for the liposarcoma. In some embodiments, the treatment for the liposarcoma comprises surgery, radiation, or both in any order.

In some embodiments, the composite gene signature score is indicative of non-liposarcoma, and the method further comprises administering a treatment to the patient appropriate for the non-liposarcoma. The non-liposarcoma may be a sarcoma or non-sarcoma. In some embodiments, the non-liposarcoma is a sarcoma, and the treatment comprises chemotherapy, radiation, surgery, or a combination of two or more of the foregoing in any order.

In some embodiments, the malignancy (e.g., liposarcoma or non-liposarcoma) is a sarcoma selected from among: Askin's tumor, Sarcoma botryoides, Chondrosarcoma, Ewing's, Malignant Hemangioendothelioma, Malignant Schwannoma, Osteosarcoma, Soft tissue sarcomas, including: Alveolar soft part sarcoma, Angiosarcoma, Cystosarcoma Phyllodes, Dermatofibrosarcoma protuberans (DF SP), Desmoid Tumor, Desmoplastic small round cell tumor, Epithelioid Sarcoma, Extraskeletal chondrosarcoma, Extraskeletal osteosarcoma, Fibrosarcoma, Gastrointestinal stromal tumor (GIST), Hemangiopericytoma (also known as “solitary fibrous tumor”), Hemangiosarcoma (More commonly referred to as “angiosarcoma”), Kaposi's sarcoma, Leiomyosarcoma, Liposarcoma, Lymphangiosarcoma, Lymphosarcoma, Malignant fibrous histiocytoma, undifferentiated pleomorphic sarcoma, Malignant peripheral nerve sheath tumor (MPNST), Neurofibrosarcoma, Plexiform Fibrohistiocytic Tumor, Rhabdomyosarcoma, Synovial sarcoma, Undifferentiated pleomorphic sarcoma (previously referred to as malignant fibrous histiocytoma).

Optionally, the method may include carrying out immunohistochemical (IHC) staining on a sample obtained from the patient before or after said determining and analyzing.

Optionally, the method may include imaging the patient before and/or after the sample is obtained, using sonography, computed tomography (CT), or magnetic resonance imaging (MM).

Another aspect of the invention concerns a method for treating a malignancy in a patient, comprising:
carrying out the diagnostic method described herein;
administering a treatment to the patient appropriate for liposarcoma if the composite gene signature score is indicative of liposarcoma, or
administering a treatment to the patient appropriate for non-liposarcoma if the composite gene signature is indicative of non-liposarcoma.

In some embodiments of the treatment method, the two or more genes comprise or consist of 2, 3, 4, 5, 6, 7, 8, 9, 10, 11, 12, 13, 14, 15, 16, 17, 18, 19, 20, 21, 22, 23, 24, or all 25 of the foregoing genes (also listed in FIG. 1). In some embodiments, the expression levels of the two or more genes in the sample comprise two or more genes selected from among MARCH2, ADH1C, ADIPOQ, AGT, AOC3, AQP7, CD36, CDO1, CNTFR, DLC1, IDS, MEST, MICAL2, PDK4, PRR5, SSX2IP, SYN2, and TIMP4. In some embodiments, the two or more genes comprise or consist of 2, 3, 4, 5, 6, 7, 8, 9, 10, 11, 12, 13, 14, 15, 16, 17, or all 18 of the foregoing genes (also listed in FIG. 4).

In some embodiments of the treatment method, the statistical method used in the diagnostic and treatment methods is selected from among Principal Component Analysis (PCA), clustering analysis, or multivariate regression analysis to generate the composite gene signature score. In some embodiments, the analysis comprises performing Principal Component Analysis (PCA) on the expression levels to obtain a first Principal Component score (PC1), wherein a PC1 less than zero is indicative of liposarcoma.

In some embodiments of the treatment method, the composite gene signature score is indicative of liposarcoma, and the method further comprises administering a treatment to the patient appropriate for the liposarcoma. In some embodiments, the treatment for the liposarcoma comprises surgery, radiation, or both in any order.

In some embodiments of the treatment method, the composite gene signature score is indicative of non-liposarcoma, and the method further comprises administering a treatment to the patient appropriate for the non-liposarcoma. The non-liposarcoma may be a sarcoma or non-sarcoma. In some embodiments, the non-liposarcoma is a sarcoma, and the treatment comprises chemotherapy, radiation, surgery, or a combination of two or more of the foregoing in any order.

Examples of chemotherapeutic agents that may be used in chemotherapy for sarcomas in the diagnostic and treatmen methods of the invention include ifosfamide, doxorubicin, cisplatin, dacarbazine (DTIC), docetaxel, gemcitabine, methotrexate, oxaliplatin, paclitaxel, vincristine, vinorelbine, trabectedin, and eribulin. The chemotherapeutic agent may be administered by itself or in combination with other chemotherapeutic agents or other agents. For example, a combination treatment is MAID (mesna, Adriamycin® (doxorubicin), ifosfamide, and dacarbazine).

Another aspect of the invention concerns an array comprising oligonucleotides attached to a surface of a support, and having specificity for a plurality of genes comprising two or more genes from among: Iduronate 2-Sulfatase (IDS), Ras Related Dexamethasone Induced 1 (RASD1), Proline Rich 5 (PRR5), Alcohol Dehydrogenase 1C (Class I): Gamma Polypeptide (ADH1C), Membrane Associated Ring-CH-Type Finger 2 (MARCH2), Family With Sequence Similarity 213 Member A (FAM213A), Centromere Protein P (CENPP), HRAS Like Suppressor Family Member 5 (HRASLS5), Synapsin II (SYN2), Ciliary Neurotrophic Factor Receptor (CNTFR), Angiotensinogen (AGT), Pyruvate Dehydrogenase Kinase 4 (PDK4), SSX Family Member 2 Interacting Protein (SSX2IP), DLC1 Rho GTPase Activating Protein (DLC1), CD36 Molecule (CD36), Adiponectin, C1Q and Collagen Domain Containing (ADIPOQ), Transmembrane Protein 132C (TMEM132C), Tissue Inhibitor of Metalloproteinases 4 (TIMP4), Butyrophilin Like 9 (BTNL9), Cysteine Dioxygenase Type 1 (CDO1), Aquaporin 7 (AQP7), Amine Oxidase, Copper Containing 3 (AOC3), Uncharacterized LOC100506100 (LOC100506100), Mesoderm Specific Transcript (MEST), and Microtubule Associated Monooxygenase, Calponin and LIM Domain Containing 2 (MICAL2) (also listed in FIG. 1). The plurality of genes may comprise or consist of two or more of the recited genes. In some embodiments, the two or more genes comprise or consist of 2, 3, 4, 5, 6, 7, 8, 9, 10, 11, 12, 13, 14, 15, 16, 17, 18, 19, 20, 21, 22, 23, 24, or all 25 of the foregoing genes (also listed in FIG. 1).

In some embodiments, the two or more genes are selected from among MARCH2, ADH1C, ADIPOQ, AGT, AOC3, AQP7, CD36, CDO1, CNTFR, DLC1, IDS, MEST, MICAL2, PDK4, PRR5, SSX2IP, SYN2, and TIMP4 (also listed in FIG. 4). The plurality of genes may comprise or consist of two or more of the recited genes. In some embodiments, the two or more genes comprise or consist of 2, 3, 4, 5, 6, 7, 8, 9, 10, 11, 12, 13, 14, 15, 16, 17, or all 18 of the foregoing genes (also listed in FIG. 4).

The arrays of the invention may be used for determining gene expression in the diagnostic methods and treatment methods of the invention by contacting a sample from the patient with the array.

In some embodiments, the plurality of genes includes no additional genes. In other embodiments, the plurality of genes includes additional genes.

In some embodiments, the plurality of genes includes additional genes, and the additional genes that are represented do not exceed 50% of the array. In some embodiments, the additional genes that are represented do not exceed 1% of the array.

In some embodiments, the oligonucleotides have specificity for less than 50 genes in total. In some embodiments, the oligonucleotides have specificity for less than 100 genes in total. In some embodiments, the oligonucleotides have specificity for less than 500 genes in total. In some embodiments, the oligonucleotides have specificity for less than 1,000 genes in total.

The arrays may include one or more probes per gene.

The arrays may include probes for additional genes. In embodiments in which the arrays include probes for additional genes genes not listed in (1), (2), or (3), above, in certain embodiments the number % of additional genes that are represented does not exceed about 50%, 40%, 30%, 20%, 15%, 10%, 8%, 6%, 5%, 4%, 3%, 2% or 1%.

In some embodiments, the arrays of the invention have oligonucleotides corresponding to DNA or RNA of less than 50, less than 100, less than 500, less than 600, less than 700, less than 800, less than 900, or less than 1000 different genes.

Microarrays are used to measure the expression levels of large numbers of genes simultaneously or to genotype multiple regions of a genome. Each nucleic acid spot typically contains picomoles (10-12 moles) of a specific oligonucleotide (e.g., DNA or RNA), known as probes (or capture probes or reporters or oligos). These can be a short section of a gene or other nucleic acid element that are used to hybridize a cDNA or cRNA (also called anti-sense RNA) sample (called target) under high-stringency conditions. Probe-target hybridization is usually detected and quantified by detection of fluorophore-, silver-, or chemiluminescence-labeled targets to determine relative abundance of nucleic acid sequences in the target.

The core principle behind microarrays is hybridization between two nucleic acid strands, the property of complementary nucleic acid sequences to specifically pair with each other by forming hydrogen bonds between complementary nucleotide base pairs. A high number of complementary base pairs in a nucleotide sequence means tighter non-covalent bonding between the two strands. After washing off non-specific bonding sequences, only strongly paired strands will remain hybridized. Labeled (e.g., fluorescently labeled) target sequences that bind to a probe sequence generate a signal that depends on the hybridization conditions (such as temperature), and washing after hybridization. Total strength of the signal, from a spot (feature), depends upon the amount of target sample binding to the probes present on that spot. Microarrays use relative quantitation in which the intensity of a feature is compared to the intensity of the same feature under a different condition, and the identity of the feature is known by its position.

Many types of arrays exist and may be utilized. The traditional solid-phase array is a collection of orderly microscopic “spots”, called features, each with thousands of identical and specific probes attached to a solid surface, such as glass, plastic or silicon biochip (commonly known as a genome chip, DNA chip or gene array). Thousands of these features can be placed in known locations on a single microarray.

Microarrays can be used to detect DNA (as in comparative genomic hybridization), or detect RNA (most commonly as cDNA after reverse transcription) that may or may not be translated into proteins. The process of measuring gene expression via cDNA is called expression analysis or expression profiling.

In certain embodiments, the arrays of the claimed invention comprise, consist, or consist essentially of oligonucleotides corresponding to mRNAs corresponding to proteins encoded by the indicated RNAs. Preferably, the oligonucleotides, which act as capture probes, are attached to a semi-solid or solid surface (e.g., plate, flow channel, bead or other particle, etc.) of glass, plastic, silicon, or other suitable material. Methods for attaching oligonucleotides to surfaces are known and may be utilized (see Seliger H et al., Curr Pharm Biotechnol, 2003, 4(6):379-395; and Beaucage S L, Curr Med Chem, 2001, 8(10):1213-1244). The oligonucleotides may be systematically arranged in different pre-determined positions, such as a grid with rows and columns (e.g., by spatial mapping or by differential tagging).

The arrayed oligonucleotide sequences are then hybridized with isolated nucleic acids (such as cDNA, miRNA or mRNA) from the test sample obtained from a subject. In some embodiments, the isolated nucleic acids from the test sample are labeled, such that their hybridization with the specific complementary oligonucleotide on the array can be determined. Alternatively, the test sample nucleic acids are not labeled, and hybridization between the oligonucleotides on the array and the target nucleic acid is detected using a sandwich assay, for example using additional oligonucleotides complementary to the target that are labeled.

In one embodiment, the hybridized nucleic acids are detected by detecting one or more labels attached to the sample nucleic acids or attached to a nucleic acid probe that hybridizes directly or indirectly to the target nucleic acids. The labels can be incorporated by any of a number of methods. In one example, the label is simultaneously incorporated during the amplification step in the preparation of the sample nucleic acids. Thus, for example, polymerase chain reaction (PCR) with labeled primers or labeled nucleotides will provide a labeled amplification product. In one embodiment, transcription amplification using a labeled nucleotide (such as fluorescein-labeled UTP and/or CTP) incorporates a label into the transcribed nucleic acids).

Detectable labels suitable for use in embodiments throughout this disclosure include any composition detectable by spectroscopic, photochemical, biochemical, immunochemical, electrical, optical or chemical means. Useful labels include biotin for staining with labeled streptavidin conjugate, magnetic beads (for example DYNABEADS), fluorescent dyes (for example, fluorescein, Texas red, rhodamine, green fluorescent protein, and the like), chemiluminescent markers, radiolabels, enzymes (for example, horseradish peroxidase, alkaline phosphatase and others commonly used in an ELISA), and colorimetric labels such as colloidal gold or colored glass or plastic (for example, polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241. In some embodiments, labels are attached by spacer arms of various lengths to reduce potential steric hindrance.

Means of detecting such labels are also well known. Thus, for example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and colorimetric labels are detected by simply visualizing the colored label.

The label may be added to the target (sample) nucleic acid(s) prior to, or after, the hybridization. So-called “direct labels” are detectable labels that are directly attached to or incorporated into the target (sample) nucleic acid prior to hybridization. In contrast, so-called “indirect labels” are joined to the hybrid duplex after hybridization. Often, the indirect label is attached to a binding moiety that has been attached to the target nucleic acid prior to the hybridization. Thus, for example, the target nucleic acid may be biotinylated before the hybridization. After hybridization, an avidin-conjugated fluorophore will bind the biotin bearing hybrid duplexes providing a label that is easily detected (see Laboratory Techniques in Biochemistry and Molecular Biology, Vol. 24: Hybridization With Nucleic Acid Probes, P. Tijssen, ed. Elsevier, N.Y., 1993).

The liposarcoma may be any subtype (e.g., well-differentiated, de-differentiated, myxoid/round cell, or pleomorphic). The non-liposarcoma may be a sarcoma or may be a cancer type other than a sarcoma.

The liposarcoma or non-liposarcoma may be a sarcoma selected from among: Askin's tumor, Sarcoma botryoides, Chondrosarcoma, Ewing's, Malignant Hemangioendothelioma, Malignant Schwannoma, Osteosarcoma, Soft tissue sarcomas, including: Alveolar soft part sarcoma, Angiosarcoma, Cystosarcoma Phyllodes, Dermatofibrosarcoma protuberans (DFSP), Desmoid Tumor, Desmoplastic small round cell tumor, Epithelioid Sarcoma, Extraskeletal chondrosarcoma, Extraskeletal osteosarcoma, Fibrosarcoma, Gastrointestinal stromal tumor (GIST), Hemangiopericytoma (also known as “solitary fibrous tumor”), Hemangiosarcoma (More commonly referred to as “angiosarcoma”), Kaposi's sarcoma, Leiomyosarcoma, Liposarcoma, Lymphangiosarcoma, Lymphosarcoma, Malignant fibrous histiocytoma, undifferentiated pleomorphic sarcoma, Malignant peripheral nerve sheath tumor (MPNST), Neurofibrosarcoma, Plexiform Fibrohistiocytic Tumor, Rhabdomyosarcoma, Synovial sarcoma, Undifferentiated pleomorphic sarcoma (previously referred to as malignant fibrous histiocytoma). Further examples of sarcomas and appropriate treatments for liposarcomas and non-liposarcomas are provided in Pham V. et al. (“Practical Issues for Retroperitoneal Sarcoma”, Cancer Control, Journal of the Moffitt Cancer Center, July 2016, Vol. 23(3):249-264), which is incorporated herein by reference in its entirety.

Examples of other cancer types are listed below in Table 1.

TABLE 1
Acute Lymphoblastic Leukemia, Adult
Acute Lymphoblastic Leukemia, Childhood
Acute Myeloid Leukemia, Adult
Acute Myeloid Leukemia, Childhood
Adrenocortical Carcinoma
Adrenocortical Carcinoma, Childhood
AIDS-Related Cancers
AIDS-Related Lymphoma
Anal Cancer
Astrocytoma, Childhood Cerebellar
Astrocytoma, Childhood Cerebral
Basal Cell Carcinoma
Bile Duct Cancer, Extrahepatic
Bladder Cancer
Bladder Cancer, Childhood
Bone Cancer, Osteosarcoma/Malignant Fibrous Histiocytoma
Brain Stem Glioma, Childhood
Brain Tumor, Adult
Brain Tumor, Brain Stem Glioma, Childhood
Brain Tumor, Cerebellar Astrocytoma, Childhood
Brain Tumor, Cerebral Astrocytoma/Malignant Glioma, Childhood
Brain Tumor, Ependymoma, Childhood
Brain Tumor, Medulloblastoma, Childhood
Brain Tumor, Supratentorial Primitive Neuroectodermal Tumors,
Childhood
Brain Tumor, Visual Pathway and Hypothalamic Glioma, Childhood
Brain Tumor, Childhood
Breast Cancer
Breast Cancer, Childhood
Breast Cancer, Male
Bronchial Adenomas/Carcinoids, Childhood
Burkitt's Lymphoma
Carcinoid Tumor, Childhood
Carcinoid Tumor, Gastrointestinal
Carcinoma of Unknown Primary
Central Nervous System Lymphoma, Primary
Cerebellar Astrocytoma, Childhood
Cerebral Astrocytoma/Malignant Glioma, Childhood
Cervical Cancer
Childhood Cancers
Chronic Lymphocytic Leukemia
Chronic Myelogenous Leukemia
Chronic Myeloproliferative Disorders
Colon Cancer
Colorectal Cancer, Childhood
Cutaneous T-Cell Lymphoma, see Mycosis Fungoides and Sézary
Syndrome
Endometrial Cancer
Ependymoma, Childhood
Esophageal Cancer
Esophageal Cancer, Childhood
Ewing's Family of Tumors
Extracranial Germ Cell Tumor, Childhood
Extragonadal Germ Cell Tumor
Extrahepatic Bile Duct Cancer
Eye Cancer, Intraocular Melanoma
Eye Cancer, Retinoblastoma
Gallbladder Cancer
Gastric (Stomach) Cancer
Gastric (Stomach) Cancer, Childhood
Gastrointestinal Carcinoid Tumor
Germ Cell Tumor, Extracranial, Childhood
Germ Cell Tumor, Extragonadal
Germ Cell Tumor, Ovarian
Gestational Trophoblastic Tumor
Glioma, Adult
Glioma, Childhood Brain Stem
Glioma, Childhood Cerebral Astrocytoma
Glioma, Childhood Visual Pathway and Hypothalamic
Skin Cancer (Melanoma)
Skin Carcinoma, Merkel Cell
Small Cell Lung Cancer
Small Intestine Cancer
Soft Tissue Sarcoma, Adult
Soft Tissue Sarcoma, Childhood
Squamous Cell Carcinoma, see Skin Cancer (non-Melanoma)
Squamous Neck Cancer with Occult Primary, Metastatic
Stomach (Gastric) Cancer
Stomach (Gastric) Cancer, Childhood
Supratentorial Primitive Neuroectodermal Tumors, Childhood
T-Cell Lymphoma, Cutaneous, see Mycosis Fungoides and Sézary
Syndrome
Testicular Cancer
Thymoma, Childhood
Thymoma and Thymic Carcinoma
Thyroid Cancer
Thyroid Cancer, Childhood
Transitional Cell Cancer of the Renal Pelvis and Ureter
Trophoblastic Tumor, Gestational
Unknown Primary Site, Carcinoma of, Adult
Unknown Primary Site, Cancer of, Childhood
Unusual Cancers of Childhood
Ureter and Renal Pelvis, Transitional Cell Cancer
Urethral Cancer
Uterine Cancer, Endometrial
Uterine Sarcoma
Vaginal Cancer
Visual Pathway and Hypothalamic Glioma, Childhood
Vulvar Cancer
Waldenström's Macroglobulinemia
Wilms' Tumor
Hairy Cell Leukemia
Head and Neck Cancer
Hepatocellular (Liver) Cancer, Adult (Primary)
Hepatocellular (Liver) Cancer, Childhood (Primary)
Hodgkin's Lymphoma, Adult
Hodgkin's Lymphoma, Childhood
Hodgkin's Lymphoma During Pregnancy
Hypopharyngeal Cancer
Hypothalamic and Visual Pathway Glioma, Childhood
Intraocular Melanoma
Islet Cell Carcinoma (Endocrine Pancreas)
Kaposi's Sarcoma
Kidney (Renal Cell) Cancer
Kidney Cancer, Childhood
Laryngeal Cancer
Laryngeal Cancer, Childhood
Leukemia, Acute Lymphoblastic, Adult
Leukemia, Acute Lymphoblastic, Childhood
Leukemia, Acute Myeloid, Adult
Leukemia, Acute Myeloid, Childhood
Leukemia, Chronic Lymphocytic
Leukemia, Chronic Myelogenous
Leukemia, Hairy Cell
Lip and Oral Cavity Cancer
Liver Cancer, Adult (Primary)
Liver Cancer, Childhood (Primary)
Lung Cancer, Non-Small Cell
Lung Cancer, Small Cell
Lymphoma, AIDS-Related
Lymphoma, Burkitt's
Lymphoma, Cutaneous T-Cell, see Mycosis Fungoides and Sézary
Syndrome
Lymphoma, Hodgkin's, Adult
Lymphoma, Hodgkin's, Childhood
Lymphoma, Hodgkin's During Pregnancy
Lymphoma, Non-Hodgkin's, Adult
Lymphoma, Non-Hodgkin's, Childhood
Lymphoma, Non-Hodgkin's During Pregnancy
Lymphoma, Primary Central Nervous System
Macroglobulinemia, Waldenström's
Malignant Fibrous Histiocytoma of Bone/Osteosarcoma
Medulloblastoma, Childhood
Melanoma
Melanoma, Intraocular (Eye)
Merkel Cell Carcinoma
Mesothelioma, Adult Malignant
Mesothelioma, Childhood
Metastatic Squamous Neck Cancer with Occult Primary
Multiple Endocrine Neoplasia Syndrome, Childhood
Multiple Myeloma/Plasma Cell Neoplasm
Mycosis Fungoides
Myelodysplastic Syndromes
Myelodysplastic/Myeloproliferative Diseases
Myelogenous Leukemia, Chronic
Myeloid Leukemia, Adult Acute
Myeloid Leukemia, Childhood Acute
Myeloma, Multiple
Myeloproliferative Disorders, Chronic
Nasal Cavity and Paranasal Sinus Cancer
Nasopharyngeal Cancer
Nasopharyngeal Cancer, Childhood
Neuroblastoma
Non-Hodgkin's Lymphoma, Adult
Non-Hodgkin's Lymphoma, Childhood
Non-Hodgkin's Lymphoma During Pregnancy
Non-Small Cell Lung Cancer
Oral Cancer, Childhood
Oral Cavity Cancer, Lip and Oropharyngeal Cancer
Osteosarcoma/Malignant Fibrous Histiocytoma of Bone
Ovarian Cancer, Childhood
Ovarian Epithelial Cancer
Ovarian Germ Cell Tumor
Ovarian Low Malignant Potential Tumor
Pancreatic Cancer
Pancreatic Cancer, Childhood
Pancreatic Cancer, Islet Cell
Paranasal Sinus and Nasal Cavity Cancer
Parathyroid Cancer
Penile Cancer
Pheochromocytoma
Pineoblastoma and Supratentorial Primitive Neuroectodermal Tumors,
Childhood
Pituitary Tumor
Plasma Cell Neoplasm/Multiple Myeloma
Pleuropulmonary Blastoma
Pregnancy and Breast Cancer
Pregnancy and Hodgkin's Lymphoma
Pregnancy and Non-Hodgkin's Lymphoma
Primary Central Nervous System Lymphoma
Prostate Cancer
Rectal Cancer
Renal Cell (Kidney) Cancer
Renal Cell (Kidney) Cancer, Childhood
Renal Pelvis and Ureter, Transitional Cell Cancer
Retinoblastoma
Rhabdomyosarcoma, Childhood
Salivary Gland Cancer
Salivary Gland Cancer, Childhood
Sarcoma, Ewing's Family of Tumors
Sarcoma, Kaposi's
Sarcoma, Soft Tissue, Adult
Sarcoma, Soft Tissue, Childhood
Sarcoma, Uterine
Sezary Syndrome
Skin Cancer (non-Melanoma)
Skin Cancer, Childhood

It is understood that aspects and embodiments of the invention described herein include “consisting” and/or “consisting essentially of” aspects and embodiments.

The practice of the present invention will employ, unless otherwise indicated, conventional techniques of molecular biology (including recombinant techniques), microbiology, cell biology, biochemistry, and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, periodic updates); “PCR: The Polymerase Chain Reaction”, (Mullis et al., eds., 1994); Singleton et al., Dictionary of Microbiology and Molecular Biology 2nd ed., J. Wiley & Sons (New York, N.Y. 1994), and March, Advanced Organic Chemistry Reactions, Mechanisms and Structure 4th ed., John Wiley & Sons (New York, N.Y. 1992).

Primers, oligonucleotides and polynucleotides employed in the present invention can be generated using standard techniques known in the art.

### Further Definitions

Several aspects of the invention are described below, with reference to examples for illustrative purposes only. It should be understood that numerous specific details, relationships, and methods are set forth to provide a full understanding of the invention. One having ordinary skill in the relevant art, however, will readily recognize that the invention can be practiced without one or more of the specific details or practiced with other methods, protocols, reagents, cell lines and animals. The present invention is not limited by the illustrated ordering of acts or events, as some acts may occur in different orders and/or concurrently with other acts or events. Furthermore, not all illustrated acts, steps or events are required to implement a methodology in accordance with the present invention. Many of the techniques and procedures described, or referenced herein, are well understood and commonly employed using conventional methodology by those skilled in the art.

After setting forth the invention in detail, it may be helpful to the understanding thereof to define several terms, and these are accordingly set forth in the next section, below. Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly used dictionaries, should be interpreted as having a meaning that is consistent with their meaning in the context of the relevant art and/or as otherwise defined herein.

The terminology used herein is for the purpose of describing particular embodiments only and is not intended to be limiting of the invention. As used herein, the indefinite articles “a”, “an” and “the” should be understood to include plural reference unless the context clearly indicates otherwise.

The phrase “and/or,” as used herein, should be understood to mean “either or both” of the elements so conjoined, i.e., elements that are conjunctively present in some cases and disjunctively present in other cases.

As used herein, the terms “array”, “microarray”, “chip”, and “biochip” are interchangeable and refer to an arrangement of a collection of nucleotide sequences in a centralized location. Arrays can be on a solid substrate, such as a glass slide, or on a semi-solid substrate, such as nitrocellulose membrane. The nucleotide sequences can be DNA, RNA, or any permutations thereof. The nucleotide sequences can also be partial sequences from a gene, primers, whole gene sequences, non-coding sequences, coding sequences, published sequences, known sequences, or novel sequences.

As used herein, “or” should be understood to have the same meaning as “and/or” as defined above. For example, when separating a listing of items, “and/or” or “or” shall be interpreted as being inclusive, i.e., the inclusion of at least one, but also including more than one, of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, the terms “including”, “includes”, “having”, “has”, “with”, or variants thereof, are intended to be inclusive similar to the term “comprising.”

As used herein, the term “patient” refers to a human or non-human animal. Typically, the terms “subject”, “individual”, and “patient” may be used interchangeably herein in reference to a subject. As such, a “patient” includes a human or non-human mammal that is being treated and/or diagnosed for/with a disease, such as cancer. The term “animal,” includes, but is not limited to, mouse, rat, dog, guinea pig, cow, horse, chicken, cat, rabbit, pig, monkey, ape, chimpanzee, and human. The patient may be any age or gender. In some embodiments, the patient has a malignancy, such as liposarcoma or non-liposarcoma. The patient may be symptomatic or non-symptomatic.

The term “polynucleotide” or “nucleic acid,” as used interchangeably herein, refers to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping groups moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-2′-O-allyl, 2′-fluoro- or 2′-azido-ribose, carbocyclic sugar analogs, a-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), “(O)NR 2 (“amidate”), P(O)R, P(O)OR′, CO or CH 2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

The term “oligonucleotide,” as used herein, refers to short, single stranded polynucleotides that are at least about seven nucleotides in length and less than about 250 nucleotides in length. Oligonucleotides may be synthetic. The terms “oligonucleotide” and “polynucleotide” are not mutually exclusive. The description above for polynucleotides is equally and fully applicable to oligonucleotides.

The term “sample”, as used herein, refers to a composition that is obtained or derived from a patient that contains a cellular and/or other molecular entity that is to be characterized and/or identified, for example based on physical, biochemical, chemical and/or physiological characteristics. In some embodiments, the sample is a sample of tissue or fluid from a lipomatous lesion.

By “tissue sample” is meant a collection of similar cells obtained from a tissue of a subject. The source of the tissue sample may be solid tissue as from a fresh, frozen and/or preserved tissue sample. In one embodiment, the tissue or cell sample may be taken from a lipomatous lesion. The tissue sample may also be primary or cultured cells or cell lines taken from and/or derived from an individual. The tissue sample may contain compounds which are not naturally intermixed with the tissue in nature such as preservatives, anticoagulants, buffers, fixatives, nutrients, antibiotics, or the like.

As used herein, the term “tumor” refers to all neoplastic cell growth and proliferation, whether malignant or benign, and all pre-cancerous and cancerous cells and tissues. For example, a particular cancer may be characterized by a solid mass tumor or non-solid tumor. The solid tumor mass, if present, may be a primary tumor mass. A primary tumor mass refers to a growth of cancer cells in a tissue resulting from the transformation of a normal cell of that tissue. In most cases, the primary tumor mass is identified by the presence of a cyst, which can be found through visual or palpation methods, or by irregularity in shape, texture or weight of the tissue. However, some primary tumors are not palpable and can be detected only through medical imaging techniques such as X-rays (e.g., mammography) or magnetic resonance imaging (MRI), or by needle aspirations. The use of these latter techniques is more common in early detection. Molecular and phenotypic analysis of cancer cells within a tissue can usually be used to confirm if the cancer is endogenous to the tissue or if the lesion is due to metastasis from another site. Some tumors are unresectable (cannot be surgically removed due to, for example the number of metastatic foci or because it is in a surgical danger zone). The treatment and prognostic methods of the invention can be utilized for early, middle, or late stage disease, and acute or chronic disease.

The term “diagnosis” is used herein to refer to the identification or classification of a molecular or pathological state, disease or condition. For example, “diagnosis” may refer to identification of a particular type of sarcoma. “Diagnosis” may also refer to the classification of a particular sub-type of liposarcoma or non-liposarcoma.

The term “aiding diagnosis” is used herein to refer to methods that assist in making a clinical determination regarding the presence, degree or other nature, of a particular type of symptom or condition of cancer, such as liposarcoma or non-liposarcoma. Diagnosis of cancer, such as liposarcoma or non-liposarcoma, may be made according to any protocol that one of skill of art would use, for example, those set by the College of American Pathology.

As used herein, “treatment” refers to clinical intervention in an attempt to alter the natural course of the individual or cell being treated, and can be performed before or during the course of clinical pathology. Desirable effects of treatment include preventing the occurrence or recurrence of a disease or a condition or symptom thereof, delaying onset of the disease or condition, alleviating a condition or symptom of the disease, diminishing any direct or indirect pathological consequences of the disease, decreasing the rate of disease progression, ameliorating or palliating the disease state, and achieving remission or improved prognosis.

Accession numbers for the 25 genes listed in FIG. 1 are provided in Table 2.

TABLE 2

| Gene | GenBank Accession No. |
|---|---|
| MARCH2 | NM_001005415 /// NM_001005416 /// |
| | NM_016496 /// XM_001131240 |
| ADH1C | NM_000667 /// NM_000668 /// NM_000669 |
| ADIPOQ | NM_004797 |
| AGT | NM_000029 |
| AOC3 | NM_003734 |
| AQP7 | NM_001170 |
| CD36 | NM_000072 /// NM_001001547 /// NM_001001548 |
| CDO1 | NM_001801 |
| CNTFR | NM_001842 /// NM_147164 |
| DLC1 | NM_006094 /// NM_024767 /// NM_182643 |
| IDS | NM_000202 /// NM_006123 |
| MEST | NM_002402 /// NM_177524 /// NM_177525 |
| MICAL2 | NM_014632 |
| PDK4 | NM_002612 |
| PRR5 | NM_001017528 /// NM_001017529 /// |
| | NM_001017530 /// NM_015366 /// NM_181333 |
| SSX2IP | NM_014021 |
| SYN2 TIMP4 | NM_003178 /// NM_133625 |
| TIMP4 | NM_003256 |
| TMEM132C | XM_044062 /// XM_941994 |
| BTNL9 | NM_152547 |
| FAM213A | NM_032333 |
| HRASLS5 | NM_054108 |
| RASD1 | NM_016084 |
| CENPP | 401541 |
| LOC100506100 | NR_046240 |

Following is an example that illustrates procedures for practicing the invention. These examples should not be construed as limiting. All percentages are by weight and all solvent mixture proportions are by volume unless otherwise noted.

### Example 1—Gene Signature to Distinguish Liposarcoma from Non-Liposarcoma

Patient population and sample acquisition. A retrospective study was performed on Moffitt's prospectively collected oncology database and all patients with a diagnosis of a soft tissue sarcoma (1993-2010) were identified. Only patients who had gene expression data were included. Differentially expressed genes for liposarcoma (all types) were compared with non-liposarcoma to identify candidate genes to create a novel gene signature specific for diagnosis of liposarcoma.

TABLE 3

| Histology | # patients | Note |
|---|---|---|
| 88003 SARCOMA NOS | 3 | non-liposarcoma |
| 88013 SPINDLE CELL SARCOMA | 4 | non-liposarcoma |
| 88023 GIANT CELL SARCOMA (EXCEPT OF BONE) | 5 | non-liposarcoma |
| 88053 UNDIFFERENTIATED SARCOMA | 2 | non-liposarcoma |
| 88063 DESMOPLASTIC SMALL ROUND CELL TUMOR | 1 | non-liposarcoma |
| 88153 SOLITARY FIBROUS TUMOR MALIGNANT | 2 | non-liposarcoma |
| 88303 MALIGNANT FIBROUS HISTIOCYTOMA | 6 | non-liposarcoma |
| 88503 LIPOSARCOMA NOS | 3 | liposarcoma |
| 88513 LIPOSARCOMA WELL DIFFERENTIATED | 2 | liposarcoma |
| 88523 MYXOID LIPOSARCOMA | 8 | liposarcoma |
| 88533 ROUND CELL LIPOSARCOMA | 2 | liposarcoma |
| 88543 PLEOMORPHIC LIPOSARCOMA | 1 | liposarcoma |
| 88583 DEDIFFERENTIATED LIPOSARCOMA | 3 | liposarcoma |
| 88903 LEIOMYOSARCOMA NOS | 2 | non-liposarcoma |
| 90413 SYNOVIAL SARCOMA SPINDLE CELL | 2 | non-liposarcoma |
| 90423 SYNOVIAL SARCOMA EPITHELIOID CELL | 1 | non-liposarcoma |
| 90433 SYNOVIAL SARCOMA BIPHASIC | 1 | non-liposarcoma |
| 92313 MYXOID CHONDROSARCOMA | 1 | non-liposarcoma |
| 95403 MALIGNANT PERIPHERAL NERVE SHEATH TUMOR | 1 | non-liposarcoma |
| 96993 MARGINAL ZONE B-CELL LYMPHOMA NOS | 1 | excluded |
| total | 50 | |

Statistical Method and Analysis.

A total of 50 soft tissue sarcoma samples (19 liposarcomas and 31 non-liposarcomas) from Total Cancer Care (TCC) database were used as the training dataset. The gene expression data are contained on HuRSTA-2a520709 chips, each with 60607 probe sets for 25587 genes. Background correction, normalization, and summarizing of raw microarray data were performed using Robust Multi-array Average (RMA) algorithm implemented in Bioconductor extensions to the R statistical programming environment.

Unpaired t-tests were performed on each and every gene to identify significantly differentially expressed genes for liposarcoma vs. non-liposarcoma. The top 18 genes (p<0.01) were selected for Principal Component Analysis (PCA), a linear transformation of the variables into a lower dimensional space which retains maximal amount of information about the variables. The inventors originally developed the 25-gene signature shown in FIG. 1, however one of the external data sets used for validation contained only 18 genes thus these 18 genes were used as an 18-gene signature.

The first principal component (PC1) of the 18 genes was used as the signature. The cutoff for liposarcoma was set at PC1<0. The 18-gene signature was first self-validated on the training dataset with 18 out of 19 liposarcomas classified as “liposarcoma” (sensitivity=94.74%) and 29 out of 31 non-liposarcomas classified as “non-liposarcoma” (specificity=93 0.55%).

The signature was further validated on external datasets publicly available at GEO including: (a) GSE32569 with 6 pairs of biopsies from metastatic alveolar soft part sarcoma (ASPS) before and after Cediranib treatment and (b) GSE12972 with 19 pairs of biopsies of untreated primary cell cultures obtained from liposarcoma and doxorubicin treated cultures from the same liposarcoma.

By calculating PC1 scores using the loading factors from the training dataset and applying the cutoff to each sample in the external datasets, we classified the 12 ASPS samples as “non-liposarcoma” (specificity=100%) and 27 of 38 liposarcoma samples as “liposarcoma” (sensitivity=71.05%) [17-19].

Principal Component Analysis (PCA).

The first principal component (pc1) of the 18 genes were used as the signature. PCA is mathematically defined¹ as an orthogonal linear transformation that transforms the data to a new coordinate such that the greatest variance by some projection of the data comes to lie on the first coordinate (called the first principal component), the second greatest variance on the second coordinate, and so on. Consider a data matrix X, with column-wise zero empirical mean (the sample mean of each column has been shifted to zero), where each of the n rows represents a different sample, and each of the p columns represents a gene. Mathematically, the transformation is defined by a set of p-dimensional vectors of weights or loadings w₍ₖ₎=(w₁, wₚ)₍ₖ₎ that map each row vector xᵢ of X to a new vector of principal component scores t₍ᵢ₎=(t₁, tₘ)₍ᵢ₎, given by

tₖ₍ᵢ₎=x₍ᵢ₎·w₍ₖ₎ for i=1, . . . , n k=1, . . . , m

in such a way that the individual variables of t considered over the data set successively inherit the maximum possible variance from x, with each loading vector w constrained to be a unit vector.

The first principal component of a data vector x(,) can be given as a score t₁₍ᵢ₎=x₍ᵢ₎·w₍₁₎ in the transformed co-ordinates, where the first loading vector w₍₁₎ has to satisfy

$${w}_{( 1 )} = arg max \{ \frac{{w}^{T} {X}^{T} {X}_{w}}{{w}^{T} w} \}$$

The cutoff for liposarcoma was set at 0. Thus a PC1 less than 0 is indicative of liposarcoma while a PC1 greater than 0 is indicative of non-liposarcoma.

Conclusion.

A total of 100 samples were evaluated and 86 cases were correctly classified with an overall accuracy of 86%. The gene signature is not significantly correlated with overall survival or 5-year survival. The initial findings are promising toward the use of this gene signature as a diagnostic adjunct, especially in small biopsy specimens which would be insufficient in other molecular tests.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

U.S. Pat. No. 3,817,837
U.S. Pat. No. 3,850,752
U.S. Pat. No. 3,939,350
U.S. Pat. No. 3,996,345
U.S. Pat. No. 4,277,437
U.S. Pat. No. 4,275,149
U.S. Pat. No. 4,366,241
1. Goldblum J R, Folpe A L, Weiss S W, Enzinger F M, Weiss S W. Enzinger and Weiss's soft tissue tumors. 6th ed. Philadelphia, Pa.: Saunders/Elsevier; 2014:xiv, 1155 p.
2. Fletcher C D M, World Health Organization., International Agency for Research on Cancer. WHO classification of tumours of soft tissue and bone. 4th ed. Lyon: IARC Press; 2013:468 p.
3. Rosai J, Akerman M, Dal Cin P et al. Combined morphologic and karyotypic study of 59 atypical lipomatous tumors. Evaluation of their relationship and differential diagnosis with other adipose tissue tumors (a report of the CHAMP Study Group). Am J Surg Pathol. 1996; 20:1182-9.
4. Mandahl N, Akerman M, Aman P et al. Duplication of chromosome segment 12q15-24 is associated with atypical lipomatous tumors: a report of the CHAMP collaborative study group. CHromosomes And MorPhology. Int j Cancer. 1996; 67:632-5.
5. Crozat A, Aman P, Mandahl N, Ron D. Fusion of CHOP to a novel RNA-binding protein in human myxoid liposarcoma. Nature. 1993; 363:640-4.
6. Fritz B, Schubert F, Wrobel G et al. Microarray-based copy number and expression profiling in dedifferentiated and pleomorphic liposarcoma. Cancer Res. 2002; 62:2993-8.
7. Singer S, Socci N D, Ambrosini G et al. Gene expression profiling of liposarcoma identifies distinct biological types/subtypes and potential therapeutic targets in well-differentiated and dedifferentiated liposarcoma. Cancer Res. 2007; 67:6626-36.
8. Boland J M, Weiss S W, Oliveira A M, Erickson-Johnson M L, Folpe A L. Liposarcomas with mixed well-differentiated and pleomorphic features: a clinicopathologic study of 12 cases. Am J Surg Pathol. 2010; 34:837-43.
9. Nielsen T O, West R B, Linn S C et al. Molecular characterisation of soft tissue tumours: a gene expression study. Lancet. 2002; 359:1301-7.
10. Segal N H, Pavlidis P, Antonescu C R et al. Classification and subtype prediction of adult soft tissue sarcoma by functional genomics. Am J Pathol. 2003; 163:691-700.
11. Shimoji T, Kanda H, Kitagawa T et al. Clinico-molecular study of dedifferentiation in well-differentiated liposarcoma. Biochem Biophys Res Commun. 2004; 314:1133-40.
12. Lal A, Panos R, Marjanovic M et al. A gene expression profile test for the differential diagnosis of ovarian versus endometrial cancers. Oncotarget. 2012; 3:212-23.
13. Pillai R, Deeter R, Rigl C T et al. Validation and reproducibility of a microarray-based gene expression test for tumor identification in formalin-fixed, paraffin-embedded specimens. J Mol Dia. 2011; 13:48-56.
14. Bammler T, Beyer R P, Bhattacharya S et al. Standardizing global gene expression analysis between laboratories and across platforms. Nat Methods. 2005; 2:351-6.
15. Dobbin K K, Beer D G, Meyerson M et al. Interlaboratory comparability study of cancer gene expression analysis using oligonucleotide microarrays. Clin Cancer Res. 2005; 11:565-72.
16. Irizarry R A, Warren D, Spencer F et al. Multiple-laboratory comparison of microarray platforms. Nat Methods. 2005; 2:345-50.
17. Bioconductor. Open Source Software for Bioinformatics; 2016.
18. Institute NCIaNHGR. The Cancer Genome Atlas; 2016.
19. Omnibus G E. 2016

1. A method for distinguishing between liposarcoma and non-liposarcoma in a patient, comprising:
determining expression levels of two or more genes in a sample obtained from the subject, wherein the two or more genes comprise two or more genes from among: Iduronate 2-Sulfatase (IDS), Ras Related Dexamethasone Induced 1 (RASD1), Proline Rich 5 (PRR5), Alcohol Dehydrogenase 1C (Class I): Gamma Polypeptide (ADH1C), Membrane Associated Ring-CH-Type Finger 2 (MARCH2), Family With Sequence Similarity 213 Member A (FAM213A), Centromere Protein P (CENPP), HRAS Like Suppressor Family Member 5 (HRASLS5), Synapsin II (SYN2), Ciliary Neurotrophic Factor Receptor (CNTFR), Angiotensinogen (AGT), Pyruvate Dehydrogenase Kinase 4 (PDK4), SSX Family Member 2 Interacting Protein (SSX2IP), DLC1 Rho GTPase Activating Protein (DLC1), CD36 Molecule (CD36), Adiponectin, C1Q and Collagen Domain Containing (ADIPOQ), Transmembrane Protein 132C (TMEM132C), Tissue Inhibitor of Metalloproteinases 4 (TIMP4), Butyrophilin Like 9 (BTNL9), Cysteine Dioxygenase Type 1 (CDO1), Aquaporin 7 (AQP7), Amine Oxidase, Copper Containing 3 (AOC3), Uncharacterized LOC100506100 (LOC100506100), Mesoderm Specific Transcript (MEST), and Microtubule Associated Monooxygenase, Calponin and LIM Domain Containing 2 (MICAL2);
analyzing the determined expression levels to generate a composite gene signature score that is indicative of liposarcoma or non-liposarcoma.

2. The method of claim 1, wherein the expression levels of the two or more genes in the sample comprise two or more genes selected from among MARCH2, ADH1C, ADIPOQ, AGT, AOC3, AQP7, CD36, CDO1, CNTFR, DLC1, IDS, MEST, MICAL2, PDK4, PRR5, SSX2IP, SYN2, and TIMP4.

3. The method of claim 1, wherein the sample is a tissue sample.

4. The method of claim 1, wherein the sample is from a lipomatous lesion.

5. The method of claim 4, wherein the lipomatous lesion is a tumor.

6. The method of claim 1, wherein said analyzing comprises Principal Component Analysis (PCA), clustering analysis, or multivariate regression analysis to generate the composite gene signature score.

7. The method of claim 1, wherein said analyzing comprises performing Principal Component Analysis (PCA) on the expression levels to obtain a first Principal Component score (PC1), wherein a PC1 less than zero is indicative of liposarcoma.

8. The method of claim 1, wherein the composite gene signature score is indicative of liposarcoma, and wherein the method further comprises administering a treatment to the patient appropriate for the liposarcoma.

9. The method of claim 8, wherein the treatment comprises surgery, radiation, or both in any order.

10. The method of claim 1, wherein the composite gene signature score is indicative of non-liposarcoma, and wherein the method further comprises administering a treatment to the patient appropriate for the non-liposarcoma.

11. The method of claim 10, wherein the non-liposarcoma is a sarcoma.

12. The method of claim 1, wherein the liposarcoma or non-liposarcoma is a sarcoma is selected from among: Askin's tumor, Sarcoma botryoides, Chondrosarcoma, Ewing's, Malignant Hemangioendothelioma, Malignant Schwannoma, Osteosarcoma, Soft tissue sarcomas, including: Alveolar soft part sarcoma, Angiosarcoma, Cystosarcoma Phyllodes, Dermatofibrosarcoma protuberans (DF SP), Desmoid Tumor, Desmoplastic small round cell tumor, Epithelioid Sarcoma, Extraskeletal chondrosarcoma, Extraskeletal osteosarcoma, Fibrosarcoma, Gastrointestinal stromal tumor (GIST), Hemangiopericytoma (also known as “solitary fibrous tumor”), Hemangiosarcoma (more commonly referred to as “angiosarcoma”), Kaposi's sarcoma, Leiomyosarcoma, Liposarcoma, Lymphangiosarcoma, Lymphosarcoma, Malignant fibrous histiocytoma, undifferentiated pleomorphic sarcoma, Malignant peripheral nerve sheath tumor (MPNST), Neurofibrosarcoma, Plexiform Fibrohistiocytic Tumor, Rhabdomyosarcoma, Synovial sarcoma, Undifferentiated pleomorphic sarcoma (previously referred to as malignant fibrous histiocytoma).

13. The method of claim 10, wherein the non-liposarcoma is a cancer other than sarcoma.

14. The method of claim 10, wherein the non-liposarcoma is a sarcoma, and wherein the treatment comprises chemotherapy, radiation, surgery, or a combination of two or more of the foregoing in any order.

15. The method of claim 1, further comprising carrying out immunohistochemical (IHC) staining on a sample obtained from the patient before or after said determining and analyzing.

16. The method of claim 1, further comprising imaging the patient before and/or after the sample is obtained, using sonography, computed tomography (CT), or magnetic resonance imaging (MRI).

17. A method for treating a malignancy in a patient, comprising:
carrying out the method of claim 1;
administering a treatment to the patient appropriate for liposarcoma if the composite gene signature score is indicative of liposarcoma, or
administering a treatment to the patient appropriate for non-liposarcoma if the composite gene signature is indicative of non-liposarcoma.

18. An array comprising oligonucleotides attached to a surface of a support, and having specificity for a plurality of genes comprising two or more genes from among: Iduronate 2-Sulfatase (IDS), Ras Related Dexamethasone Induced 1 (RASD1), Proline Rich 5 (PRR5), Alcohol Dehydrogenase 1C (Class I): Gamma Polypeptide (ADH1C), Membrane Associated Ring-CH-Type Finger 2 (MARCH2), Family With Sequence Similarity 213 Member A (FAM213A), Centromere Protein P (CENPP), HRAS Like Suppressor Family Member 5 (HRASLS5), Synapsin II (SYN2), Ciliary Neurotrophic Factor Receptor (CNTFR), Angiotensinogen (AGT), Pyruvate Dehydrogenase Kinase 4 (PDK4), SSX Family Member 2 Interacting Protein (SSX2IP), DLC1 Rho GTPase Activating Protein (DLC1), CD36 Molecule (CD36), Adiponectin, C 1Q and Collagen Domain Containing (ADIPOQ), Transmembrane Protein 132C (TMEM132C), Tissue Inhibitor of Metalloproteinases 4 (TIMP4), Butyrophilin Like 9 (BTNL9), Cysteine Dioxygenase Type 1 (CDO1), Aquaporin 7 (AQP7), Amine Oxidase, Copper Containing 3 (AOC3), Uncharacterized LOC100506100 (LOC100506100), Mesoderm Specific Transcript (MEST), and Microtubule Associated Monooxygenase, Calponin and LIM Domain Containing 2 (MICAL2).

19. The array of claim 18, wherein the two or more genes are selected from among MARCH2, ADH1C, ADIPOQ, AGT, AOC3, AQP7, CD36, CDO1, CNTFR, DLC1, IDS, MEST, MICAL2, PDK4, PRR5, SSX2IP, SYN2, and TIMP4.

20. The array of claim 18, wherein the plurality of genes includes no additional genes.

21-27. (canceled)
